(19) United States
(12) Patent Application Publication
(10) Pub. No.: US 2026/0364592 A1
(43) Pub. Date: Nov. 19, 2026

(54) DEVICE FOR DISPENSING A FLUID DROP-BY-DROP

(71) Applicant: Henke-Sass, Wolf GmbH, Tuttlingen (DE)

(72) Inventors: Axel RIEDLINGER, Schoemberg (DE); Simon RAIDT, Wurmlingen (DE); Marco LEICHT, Voehringen (DE)

(21) Appl. No.: 19/679,306

(22) Filed: May 15, 2026

(30) Foreign Application Priority Data
May 15, 2025 (DE) 102025118912.9

(51) Int. Cl. B01L 3/02

(52) U.S. Cl. CPC B01L 3/0265; B01L 2200/087; B01L 2300/123; B01L 2400/0605

(57) ABSTRACT

A device for dispensing a fluid drop-by-drop includes a housing with a fluid port, through which the fluid to be dispensed can be supplied, an outlet nozzle through which the fluid can be dispensed drop-by-drop, a fluid conduit connecting the fluid port to the outlet nozzle, an underside, and an actuating element. Upon actuation of the actuating element, a predetermined amount of fluid is conveyed toward the outlet nozzle. The outlet nozzle includes a tubular end portion extending in a straight line along a first longitudinal direction with a bevelled dispensing end that has a flat end face. In an imaginary longitudinal section of the tubular end portion, the flat end face faces the underside and comprises a first angle that is less than 90°.

### PRIORITY

This application claims the benefit of German Patent Application No. 10 2025 118912.9, filed on May 15, 2025, which is hereby incorporated herein by reference in its entirety.

### FIELD

The present invention relates to a device for dispensing a fluid drop-by-drop.

### BACKGROUND

If the fluid is, for example, a vaccine, such a device can be used to dispense the vaccine drop-by-drop.

For example, it is desirable to administer eye drops to poultry for vaccination, thereby performing ocular vaccination. To date, such ocular vaccination has been performed using a plastic bottle. By applying manual pressure to the bottle wall, a droplet is formed at a dispensing tip. As soon as the droplet is released, the pressure is removed, so that the remaining vaccine is drawn back into the bottle by the resulting vacuum. Droplet size and number of droplets depend both on the pressure applied by the user and on the angle at which the bottle is held during droplet dispensing.

In practical application for the ocular vaccination of an animal, for example, it may happen that two or more droplets are dispensed, as a large number of animals typically need to be treated under significant time pressure. This results in user-related variability, which leads, on the one hand, to fluctuations in dosage and, on the other hand, to a vaccine requirement that is difficult to calculate for a quantitatively defined animal population.

Furthermore, direct contact between the user's hand and the bottle causes the medication to warm up, which can impair the vaccine's efficacy.

### SUMMARY

It is an object herein to provide a device for dispensing a fluid drop-by-drop, with which the described difficulties can be overcome as completely as possible.

A device for dispensing a fluid drop-by-drop (which may also be referred to as a drop applicator and, in particular, as a drop applicator for ocular vaccination) is provided, wherein the device comprises a housing with a fluid port through which the fluid to be dispensed can be supplied, an outlet nozzle through which the fluid can be dispensed drop-by-drop, a fluid conduit connecting the fluid port to the outlet nozzle, an underside and an actuating element. Upon actuation of the actuating element, a predetermined amount of fluid is conveyed toward the outlet nozzle (preferably for the delivery of a single droplet of fluid), wherein the outlet nozzle comprises a tubular end portion extending in a straight line along a first longitudinal direction with a bevelled dispensing end that comprises a flat (preferably annular) end face. In an imaginary longitudinal section of the tubular end portion, the flat end face faces to the underside and forms a first angle with the first longitudinal direction that is less than 90°.

When used as intended, the device can be ergonomically held and operated with one hand (without, for example, having to additionally bend the wrist during operation), wherein the flat end face of the dispensing end may face the ground. In particular, the end face may be aligned parallel to the ground surface or to the horizontal. This ensures high dosing accuracy (including repeatable dispensing of a single droplet).

This can further result in reduced variation in droplet size, since the adhesive forces of the fluid (e.g., the medication or vaccine) act only on the end face, thereby preventing wetting and in particular, increasing wetting of the outer surface of the tubular end portion.

The first angle, in the imaginary longitudinal section, can also be described as open toward the underside. Furthermore, in the imaginary longitudinal section, the bevelled dispensing end (or the chamfered dispensing end) can be characterized by the fact that the end face and the outer surface of the end portion facing the underside are arranged in a V-shape, wherein the V opens away from the underside (toward the upper side of the housing).

The fluid may be a liquid and/or a medication (such as a vaccine). The drop applicator may be used in particular for ocular vaccination and may thus be suitable for this purpose.

The first angle may be less than or equal to 85°, 80°, 75°, 70°, 65°, 60°, 55°, 50°, or 45°.

Furthermore, the first angle may be greater than or equal to 10°, 15°, 20°, 25°, 30°, 35°, 40°, 45°, 50°, 55°, 60°, 65°, 70°, 75°, 80°, or 85°. Preferably, the first angle comprises a value in the range of 40°-60° (including the limits), and more preferably in the range of 45°-55° (including the limits). The first angle may, for example, be 46°, 47°, 48°, 49°, 50°, 51°, 52°, or 53°.

The fluid port may be configured to hold a bottle (or container) containing the fluid (e.g., a medicine bottle). The fluid port is preferably configured such that the axis of the bottle is perpendicular to the plane in which the flat end face of the dispensing end lies. When the drop applicator is used as intended and the flat end face is aligned parallel to the Earth's surface, the axis of the bottle is parallel to the vertical, allowing for easy and complete emptying of the bottle.

The area of the flat end face of the dispensing end can be used to determine the droplet volume at which the droplet detaches from the dispensing end of the outlet nozzle and falls due to gravity. The larger the area of the upper end face, the greater the adhesive forces that must be overcome by gravity. Therefore, the desired drop volume can advantageously be set by means of the specified area. For example, for an average drop volume of 0.028 ml, the area of the end face is 2.9 mm².

The tubular end portion of the outlet nozzle preferably protrudes beyond the housing, so that the desired positioning of the flat end face (preferably parallel to the horizontal) can be easily achieved during droplet dispense.

Since the flat end face of the dispensing end faces downward and forms an angle of less than 90° with the first longitudinal direction, backflow of the droplet (or parts thereof) along the outer surface of the tubular end portion during droplet dispensing can additionally be prevented, which contributes to high dosing accuracy.

Thus, the device ensures that only a single droplet of a defined volume is dispensed per actuation of the actuating element. This reduces vaccine wastage.

Furthermore, the spatial separation of the fluid port (to which, for example, a reservoir or bottle containing the desired fluid, e.g., a liquid medication) and the areas of the housing where the drop applicator is held during intended use, unwanted heating of the fluid (e.g., the medication) by body heat is prevented.

The underside of the housing may extend along a second longitudinal direction, wherein, in the imaginary longitudinal section of the tubular end portion, the first longitudinal direction forms a second angle with the second longitudinal direction that is open toward the underside and less than 180°. In particular, the second angle may be less than or equal to 175°, 170°, 165°, 160°, 155°, 150°, or 145°, as well as greater than or equal to 100°, 105°, 110°, 115°, 120°, 125°, 130°, 135°, 140°, 145°, 150°, 155°, 160°, 165°, or 170°. Preferably, the second angle lies in a range of 175°-145° (including the limits), and more preferably 170°-150° (including the limits). In particular, the second angle may be 170°, 169°, 168°, 167°, 166°, 165°, 164°, 163°, 162°, 161°, 160°, 159°, 158°, 157°, 156°, or 155°.

The housing may comprise a connection section that comprises the fluid port, said connection section protrudes from the upper side of the housing. In particular, it may protrude obliquely from the upper side of the housing. The connection section may extend along a third longitudinal direction, wherein, in the imaginary longitudinal section of the tubular end portion, the third longitudinal direction forms a third angle with the flat end face within a range of 80°-100° (including the limits) and preferably within a range of 85°-95° (including the limits). Particularly preferably, the third angle is 85°, 86°, 87°, 88°, 89°, 90°, 91°, 92°, 93°, 94°, or 95°, and particularly preferably it is 90°.

The fluid conduit (or fluid connection) may comprise an elastic tube (or an elastic hose) against which the actuating element is pressed upon actuation to reduce the tube cross-section and thereby deliver the desired fluid volume for a droplet to be dispensed to the outlet nozzle.

Upon actuation of the actuating element, the latter can thus be pressed against a contact area of the elastic tube, wherein a check valve may be arranged in the elastic tube upstream of the contact area and a check valve may be arranged in the elastic tube downstream of the contact area. In particular, the two check valves may be designed such that, once a predetermined overpressure exists in the elastic tube between the two check valves, the downstream check valve opens, and once a predetermined underpressure exists in the elastic tube between the two check valves, the upstream check valve opens.

This allows the desired fluid volume for dispensing a droplet to be delivered to the outlet nozzle in a mechanically very simple and thus also cost-effective manner.

The actuating element may comprise a rod-shaped segment mounted slidably in the housing, the end of which facing the elastic tube is pressed against the elastic tube when the actuating element is actuated.

The actuating element may further comprise an end portion projecting beyond the housing (preferably beyond the upper side of the housing or the upper side of a corresponding holding section of the housing), upon which a force can be applied to press the actuating element against the tube.

The restoring force of the elastic tube may be such that the weight of the actuating element alone is insufficient to deliver fluid to the outlet nozzle.

The length of the actuator element can thus determine how much fluid volume is delivered per actuation, wherein this is matched to the surface area of the end face to reproducibly produce the desired droplet size.

If the length of the actuator element is too short, insufficient fluid volime is delivered and the droplet does not detach from the end face. However, if the length of the actuating element is too long, too much fluid volume is delivered, which can have the effect of causing the formation of a further partial droplet after the droplet detaches; this, in combination with the fluid from a subsequent actuation of the actuating element, can lead to a double droplet.

The housing may comprise two recessed grips (or two finger recesses) in the front area, facing away from one another, for the user's thumb on the one hand and middle and ring fingers on the other hand, to allow the housing to be held in the so-called three-point grip. In this position, the underside of the housing rests on the indentation on the back of the hand between the thumb and index finger, wherein the thumb and middle finger (or ring finger) rest against the finger recesses. The actuating element is preferably arranged such that, in the three-point grip, the index finger can press the actuating element against the elastic tube. Thus, the device can be held and operated with one hand, similar to a pen. The dispensing of droplets is thus possible with one hand.

The recessed grips may, in particular, be arranged on the housing such that the device is equally suitable for both right-handed and left-handed users.

The housing, the actuating element, and the outlet nozzle may be made of plastic. For the housing and the actuating element, PC and/or ABS may be used, for example. POM, for example, can be used for the outlet nozzle.

It is understood that the features mentioned above and those to be described below can be used not only in the specified combinations but also in other combinations or individually, without departing from the scope of the present invention.

The invention will now be described in more detail below with reference to the accompanying drawings, which also disclose features essential to the invention. These embodiments are provided for illustrative purposes only and should not be construed as limiting. For example, a description of an embodiment with a plurality of elements or components should not be interpreted to mean that all of these elements or components are necessary for implementation. Rather, other embodiments may also include alternative elements and components, fewer elements or components, or additional elements or components. Elements or components from different embodiments may be combined with one another unless otherwise specified. Modifications and variations described for one of the embodiments may also be applicable to other embodiments. To avoid repetition, identical or corresponding elements in different figures are designated by the same reference numerals and are not described multiple times.

### BRIEF DESCRIPTION OF THE DRAWINGS

FIG. 1 is a perspective view of an embodiment of device for dispensing a fluid drop-by-drop, wherein the left side of housing is visible.

FIG. 2 is a perspective view of device from FIG. 1, wherein the right side of housing is visible.

FIG. 3 is a view of the underside of the housing of the device according to FIG. 1.

FIG. 4 is a longitudinal section of the device according to FIG. 1.

FIG. 5 is an enlarged view of detail A from FIG. 3.

FIG. 6 is an enlarged view of detail B from FIG. 4.

FIG. 7 is a diagram illustrating the different longitudinal directions R1-R3, their orientations, and their orientations relative to the end face.

FIG. 8 is a perspective view of the device according to FIG. 1, wherein the mounting connector holds a container.

FIG. 9 is a longitudinal section view of the device with the container according to FIG. 8.

FIG. 10 is the longitudinal section view according to FIG. 9 with fluid FL shown.

### DETAILED DESCRIPTION

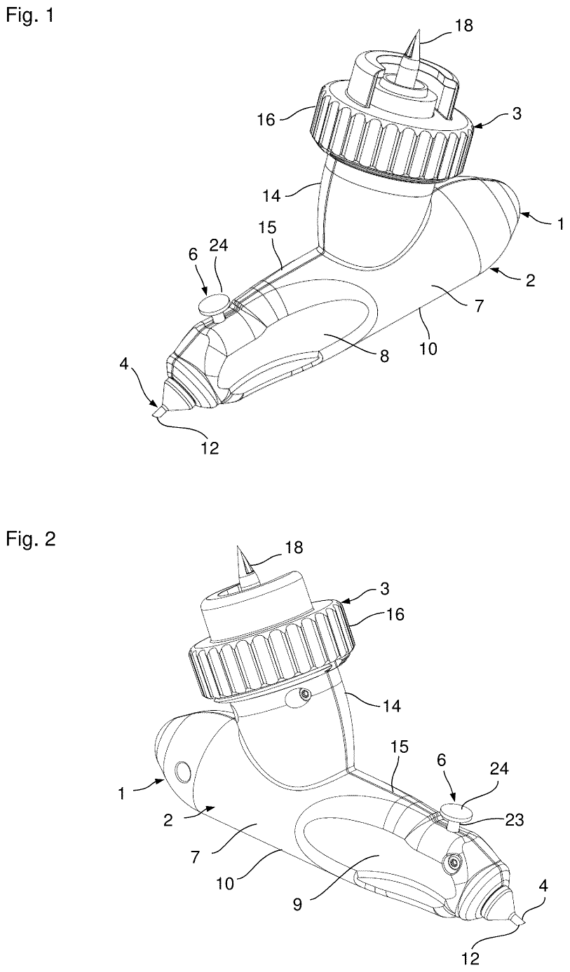

In the embodiment shown in FIGS. 1 through 10, the device 1 for dispensing a fluid drop-by-drop (which may also be referred to as a drop applicator 1) comprises a housing 2 with a fluid port 3 through which the fluid to be dispensed can be supplied, an outlet nozzle 4, through which the fluid is to be dispensed drop-by-drop, a fluid conduit 5 connecting the fluid port 3 to the outlet nozzle 4, and an actuating element 6.

The housing 2 comprises an elongated holding section 7 with two lateral finger recesses 8, 9 in the front region of the holding section 7, which are designed for a user's thumb and middle finger (or ring finger). The holding section 7 comprises an underside 10 that extends along a longitudinal direction R2. When using the device 1 as intended, the user holds the device 1 like a pen in the so-called three-point grip. In this position, the underside 10 of the housing 2 rests on the indentation on the back of the hand between the thumb and index finger, wherein the thumb and middle finger (or ring finger) rest against the finger recesses 8, 9. The index finger can then be used to press the actuating element 6 downward to dispense the desired droplet of fluid through the outlet nozzle 4, as will be described in detail below.

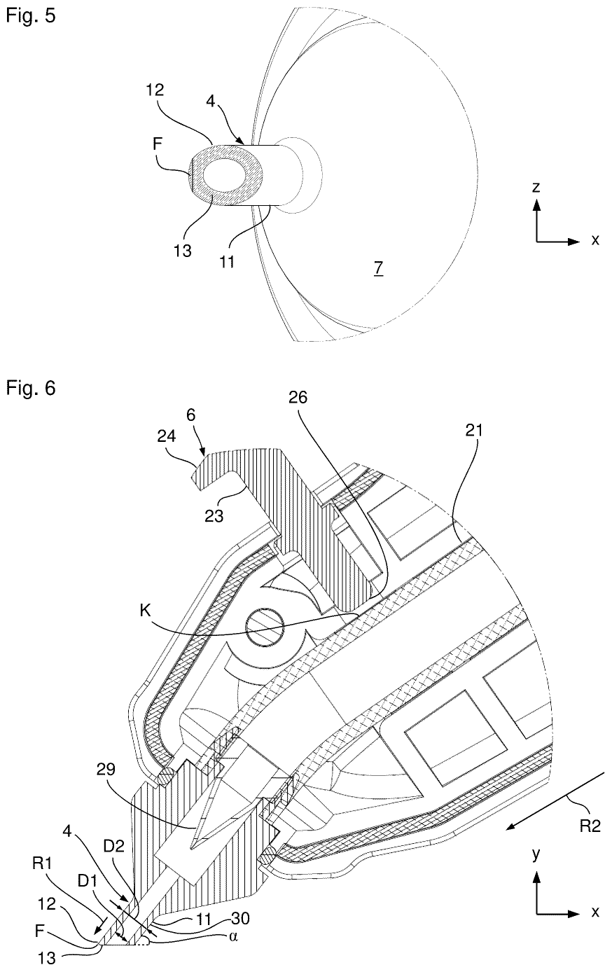

The outlet nozzle 4 comprises a tubular end portion 11 extending in a straight line along a longitudinal direction R1 with a bevelled dispensing end 12, such that a flat annular end face 13 is formed, which faces the underside 10 and forms an angle α of less than 90° with the longitudinal direction R1 (see in particular FIGS. 4, 6, and 7). The angle α is open toward the underside 10. In the illustrated embodiment, the angle α is 52°. A chamfer F (FIG. 5) may be formed on the side of the bevelled dispensing end 12 facing away from the housing 2. However, it is also possible to omit the chamfer.

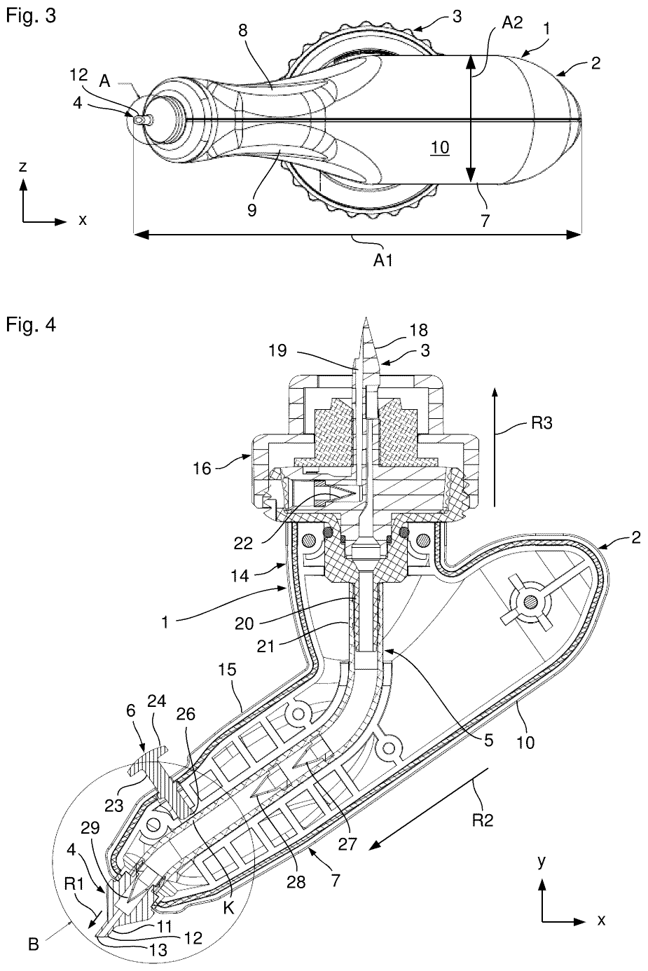

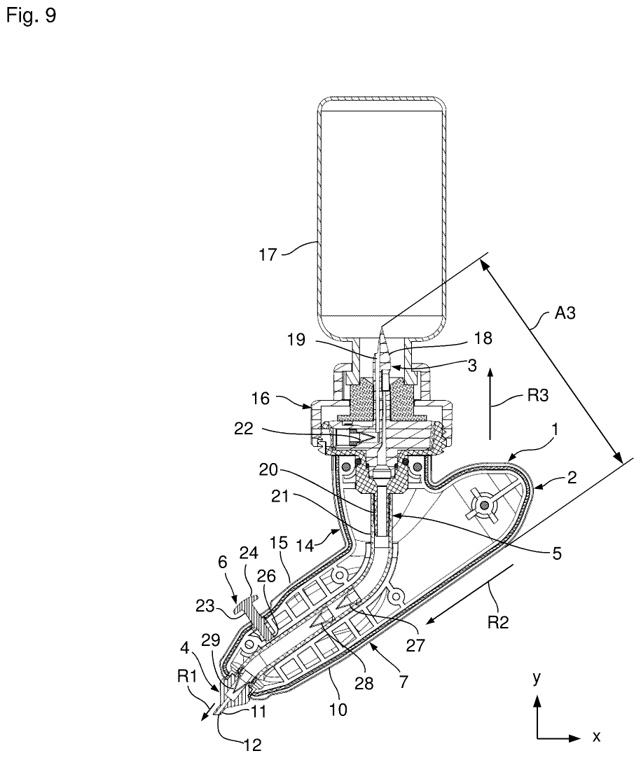

The tubular end portion 11 may comprise an inner diameter D1 of 1.1 mm and an outer diameter D2 of 1.9 mm (FIG. 6). The drop applicator 1 may comprise a height A3 of 71 mm (FIG. 9) and a length A1 of 111 mm (FIG. 3). The width A2 of the holding section 7 may be 26 mm (FIG. 3).

As shown, for example, in FIGS. 1, 2, 4, and 6, during the intended use of the drop applicator 1, the housing 2 is tilted relative to the horizontal (here indicated by the x-direction) due to the described three-point grip, such that, when holding the drop applicator 1 in this ergonomic manner, the end face 13 can be easily positioned parallel to the horizontal. As will be described below, this advantageously results, among other things, in the repeatable dispensing of a droplet containing the same amount of fluid.

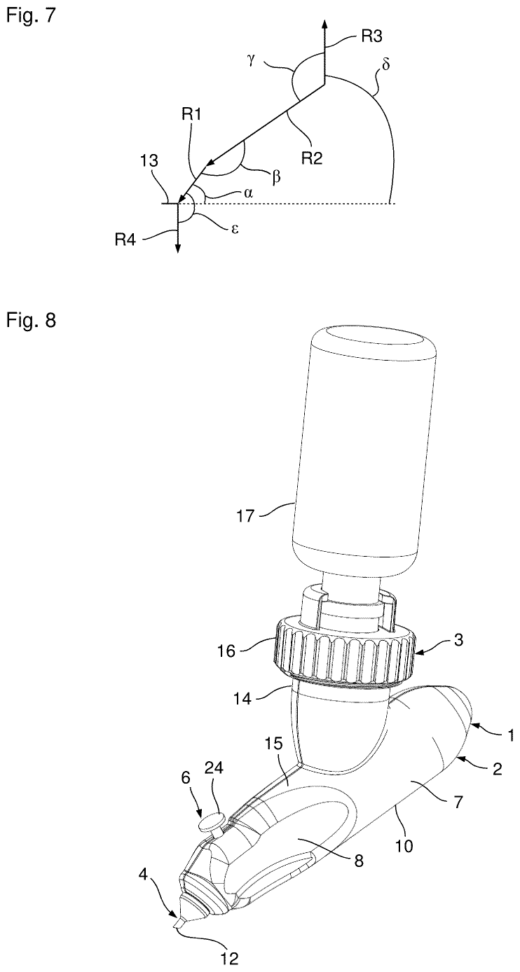

The housing 2 comprises a connection section 14 that protrudes obliquely from an upper side 15 of the holding section 7. As can be seen, for example, in FIG. 4, the connection section 14 extends along a longitudinal direction R3. The orientation of this longitudinal direction R3 is preferably chosen such that it coincides with the vertical (here the y-direction in FIG. 4) when the end face 13 is oriented parallel to the horizontal. This can be easily achieved by ensuring that, in the longitudinal section according to FIG. 4, as shown in FIG. 7, the angle δ formed by the end face 13 and the direction R3 lies in the range of 85°-95°. Preferably, the angle δ is 90°.

The connection section 14 may be positioned and formed in the middle to the rear region of the holding section 7.

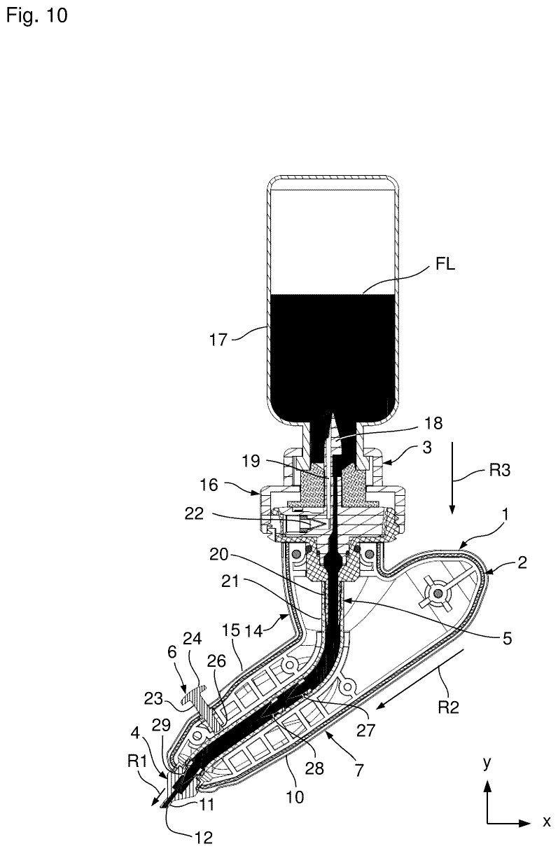

The connection section 14 comprises a connector 16 for a container 17 (as shown, for example, in FIG. 9-10) containing fluid FL to be dispensed, wherein a piercing needle 18 of the connector 16 extends into the container 17. A channel 19 is formed in the piercing needle 18, which extends further through the connector 16 to a channel end section 20 inside the housing 2. An elastic tube 21 is slipped over the channel end section 20, connecting the channel end section 20 to the outlet nozzle 4. In this manner, the fluid conduit 5 between the fluid port 3 and the outlet nozzle 4 is established.

A check valve 22 connected to the channel 19 is provided in the connection section 14, through which air can be supplied to the channel 19 to prevent a vacuum in the container 17 during discharge of the fluid FL.

As seen in the longitudinal section according to FIGS. 4 and 6, the described angle α is thus present. It can also be said that, as seen in this longitudinal section according to FIGS. 4 and 6, the flat end face 13 and the outer surface 30 facing the underside 10 are arranged in a V-shape, wherein the V is open away from the underside 10 and toward the upper side 15.

As can be seen in particular from FIGS. 4 and 6, in the front region of the upper side 15, the actuating element 6 is formed as a slidably mounted rod-shaped segment 23, which has an end portion 24 (here referred to as contact point 24) and comprises its opposite end 26 toward a contact area K on the tube 21. Between the contact area K and the connector 16 (and thus upstream), two check valves 28 and 29 are arranged within the tube 21, and between the contact area K and the outlet nozzle 4 (and thus downstream), a further check valve 29 is arranged.

As can be clearly seen, for example, in FIGS. 4, 6, and 7, the tubular end portion 11 is inclined relative to the longitudinal direction R2 along which the elastic tube 21 (and thus the fluid channel 5) extends in the region of the actuating element 6. In the embodiment described here, the two longitudinal directions R1 and R2 form an angle β open toward the underside 10, which here is 163°. To arrive at the values for the angle δ specified above, the longitudinal directions R2 and R3 form an angle γ open toward the upper side, which here is 125°. This results in an angle ε of 142° between the longitudinal direction R1 and a perpendicular R4 to the end face 13. As previously stated, during the intended use of the drop applicator 1, it is held such that the flat end face 13 is positioned parallel to the horizontal, so that the perpendicular R4 in this case corresponds to the direction along which the respective droplet is dispensed.

The housing 2 may be made of PC/ABS (polycarbonate/acrylonitrile-butadiene-styrene). The outlet nozzle 4 may be made of POM (polyoxymethylene).

If the actuator 6 is now pressed against the elastic tube 21 with the index finger by a user holding the device 1 in the three-point grip, an overpressure is generated in the tube 21 between the check valves 28 and 29, causing the check valve 29 to open (whereas check valves 27 and 28 remain locked) and the desired amount of fluid is dispensed as a droplet via the dispensing end 12. When pressure (e.g., from the index finger) is no longer applied to the actuating element 6, it is moved toward the upper side 15 due to the restoring force of the elastic tube 21, causing check valve 29 to close; the resulting negative pressure between check valve 29 and check valve 28 causes check valves 27 and 28 to open, allowing fluid FL to flow from container 17 into the area between check valves 28 and 29.

When a container 17 containing fluid FL is connected to the connector 16 and no fluid

FL is yet present in the fluid conduit 5 from the connector 16 to the outlet nozzle 4, or the fluid conduit 5 is not completely filled with fluid FL, the actuating element 6 is actuated once or several times for filling, until fluid FL is visible at the discharge end 12 of the outlet nozzle 4. The device 1 is then ready for use, and any further actuation of the actuating element 6 results in the discharge of the desired drop of fluid.

The provision of two check valves 27 and 28 is not mandatory. A single check valve is also sufficient. This depends on the design of the device 1 and the characteristics of the check valves 27-29. In the present embodiment, standard check valves were used, so that the two check valves 27 and 28 had to be arranged between the connection section 14 and the actuating element 6.

The holding section 7 may be designed such that the drop applicator 1 can be operated with either the right hand or the left hand. When held with the left hand, the thumb rests on the finger recess 9 and the middle finger (or ring finger) rests on the finger recess 8.

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products. Moreover, features or aspects of various example embodiments may be mixed and matched (even if such combination is not explicitly described herein) without departing from the scope of the invention.

What is claimed is:

1. A device for dispensing a fluid drop-by-drop, comprising:
a housing, comprising:
a fluid port, through which the fluid to be dispensed can be supplied,
an outlet nozzle through which the fluid can be dispensed drop-by-drop,
a fluid conduit connecting the fluid port to the outlet nozzle,
an underside, and
an actuating element,
wherein a predetermined amount of fluid is conveyed towards the outlet nozzle upon actuation of the actuating element,
wherein the outlet nozzle comprises a tubular end portion extending in a straight line along a first longitudinal direction with a bevelled discharge end that comprises a flat end face, and
wherein, in an imaginary longitudinal section of the tubular end portion, the flat end face faces the underside and forms a first angle with the first longitudinal direction that is less than 90°.

2. The device according to claim 1, wherein the underside of the housing extends along a second longitudinal direction, and wherein, in the imaginary longitudinal section of the tubular end portion, the first longitudinal direction forms a second angle with the second longitudinal direction that is open toward the underside and less than 180°.

3. The device according to claim 2, wherein the housing comprises a connection section having the fluid port, said connection section protruding from the upper side of the housing and extending along a third longitudinal direction, and wherein, in the imaginary longitudinal section of the tubular end portion, the third longitudinal direction forms a third angle with the flat end face within a range of 85° to 95°.

4. The device according to claim 3, wherein the connection section protrudes obliquely from the upper side of the housing.

5. The device according to claim 1, wherein the housing comprises a connection section having the fluid port, said connection section protruding from the upper side of the housing and extending along a third longitudinal direction, and wherein, in the imaginary longitudinal section of the tubular end portion, the third longitudinal direction forms a third angle with the flat end face within a range of 85° to 95°.

6. The device according to claim 5, wherein the connection section protrudes obliquely from the upper side of the housing.

7. The device according to claim 1, wherein the fluid conduit comprises a flexible tube against which the actuating element is pressed upon actuation to reduce a tube cross-section of the tube and thereby deliver the desired fluid volume to the outlet nozzle.

8. The device according to claim 7,
wherein the actuating element is pressed against a contact area of the elastic tube upon actuation,
wherein a check valve is arranged in the elastic tube both upstream and downstream of the contact area, respectively,
wherein, once a predetermined overpressure exists between the upstream and downstream check valves, the downstream check valve opens, and once a predetermined underpressure exists in the elastic tube between the upstream and downstream check valves, the upstream check valve opens.

9. The device according to claim 8, wherein the actuating element comprises a rod-shaped segment slidably mounted in the housing, the end of which facing the elastic tube is pressed against the elastic tube.

10. The device according to claim 8, wherein the actuating element comprises an end portion projecting beyond the housing, upon which a force can be applied to press the actuating element against the elastic tube.

11. The device according to claim 8, wherein a restoring force of the elastic tube is such that the weight of the actuating element alone is insufficient to convey fluid to the outlet nozzle.

12. The device according to claim 7, wherein the actuating element comprises a rod-shaped segment slidably mounted in the housing, the end of which facing the elastic tube is pressed against the elastic tube.

13. The device according to claim 7, wherein the actuating element comprises an end portion projecting beyond the housing, upon which a force can be applied to press the actuating element against the elastic tube.

14. The device according to claim 7, wherein a restoring force of the elastic tube is such that the weight of the actuating element alone is insufficient to convey fluid to the outlet nozzle.

15. The device according to claim 1, wherein the housing comprises, in the front region, two recessed grips facing away from each other for a user's thumb on one side and middle finger or ring finger on the other side, to enable the housing to be held in a three-point grip.

16. The device according to claim 1, wherein the first angle is in the range of 40° to 60°.

17. The device according to claim 1, wherein the tubular end portion protrudes beyond the housing.
